(19) United States
(12) Patent Application Publication
(10) Pub. No.: US 2007/0166307 A1
(43) Pub. Date: Jul. 19, 2007

(54) Suppression of transplant rejection

(75) Inventors: Andrew Bushell, Oxford (GB); Kathryn Wood, Oxford (GB); Mahzuz Karim, Oxford (GB); Vanessa Oliveira, Oxford (GB); Brigit Sawitzki, Oxford (GB)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ISIS Innovation Limited, Oxford (GB)

(21) Appl. No.: 10/561,411

(22) PCT Filed: Jun. 18, 2004
(86) PCT No.: PCT/GB04/02647

§ 371 Date: Feb. 14, 2007

(30) Foreign Application Priority Data
Jun. 20, 2003 (GB) 0314461.5

(51) Int. Cl. A61K 39/395; C12N 5/08

(52) U.S. Cl. 424/144.1; 435/325; 435/372

(57) ABSTRACT

The present invention relates to a transplant rejection in an animal suppressed by administration of an antibody directed at a cell surface antigen selected from the group consisting of CD4, CD8, CD154, LFA-1, CD80, CD86 and ICAM-1, preferably an anti-CD4 antibody, together with a non-cellular protein antigen to generate in the animal a population of regulatory T-lymphocytes; reactivating said population of regulatory T-lymphocytes by further administration to the animal of the non-cellular protein antigen; and transplanting said organ or tissue whilst said population of regulatory T-lymphocytes is activated. Regulatory T cells can be generated ex vivo by culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen. Ex vivo generated T-lymphocytes can be used as an alternative method of overcoming transplant rejection or in combination with the in vivo method. A similar approach can be adopted for the treatment of autoimmune conditions.

### FIELD OF THE INVENTION

This invention relates to the suppression of rejection of transplants in animals.

### BACKGROUND OF THE MOTION

Transplantation is the treatment of choice for end stage kidney, heart, liver and pancreas organ failure and despite considerable advances in the management of transplant rejection in recent years the vast majority of transplants are eventually rejected. In addition, the current immunosuppressive regimens which depend on continual drug therapy predispose transplant patients to increased susceptibility to infections and cancer because even the most sophisticated drugs are unable to inhibit just those responses directed toward the transplant. As a result opportunistic infection remains one of the main causes of mortality in heart transplant patients and predictive calculations have shown that 30 years of continual immunosuppression carries a 100% risk of some types of cancer. In many animal transplant models it is possible to achieve indefinite transplant survival by transient manipulation of the recipient immune system and in many of these situations regulatory cells develop with time such that they prevent rejection even after cessation of the initial therapy. Waldmann and Cobbold⁴⁶ discuss the developments over recent years that have led to the possibility of providing short-term therapy for long-term tolerance of organ grafts.

CD4+ T-helper lymphocytes are cells of the immune system and in normal situations play an essential role in immune responses that protect us from pathogenic organisms such as bacteria and viruses. In the context of transplantation however, these same cells are largely responsible for the rejection of organ transplants. It is widely known that rejection responses can be attenuated by administration of immunosuppressive agents, including anti-CD4 antibody which targets CD4+ T cells, but in recent years it has been shown that such antibody therapy can lead to the generation of sub-populations of T cells with the capacity to control or regulate destructive rejection responses. It is believed that regulatory cells arise in such situations because the presence of the anti-CD4 antibody prevents fill T cell activation and the cells default to a regulatory or suppressive phenotype.

EP-A-0 240 344 describes the use of a monoclonal antibody directed against the CD4 antigen on helper T lymphocytes for the manufacture of a medicament for the treatment of a mammal to induce tolerance in the said mammal to a primary antigen, the treatment comprising administering to the subject mammal sufficient of the medicament to deplete significantly the population of T-helper lymphocytes in the subject mammal, challenging the subject mammal with the primary antigen and allowing the population of T-helper lymphocytes in the subject mammal to re-establish itself in the presence of the primary antigen so that tolerance to the primary antigen is established.

EP-A-0 474 691 describes how non-depleting CD4 antibodies, optionally together with CD8 antibodies, can produce tolerance to foreign immunoglobulins, bone marrow and skin grafts. Specifically a state of immunological tolerance to an antigen can be induced by the administration of these antibodies in the presence of said antigen. Such antigens are usually foreign cellular antigens, but tolerance to soluble non-cellular antigens such as albumin³⁴ or human gamma globulin (HGG)^{35,36} has also been achieved by intravenous administration to mice under the cover of anti-CD4 antibody.

The existence of lymphocytes with suppressive capacity was first described over thirty years ago¹, but in recent years there has been renewed interest in the identification and characterisation of such regulatory T cells (T-reg). Several cell surface markers have been identified that enrich for regulatory activity, one of which is CD25, the α subunit of the IL-2 receptor.

CD25⁺CD4⁺T-reg with the capacity to regulate responses in vitro have been identified in both mice²⁻⁶ and humans⁷⁻¹². T-reg can suppress the proliferation and/or effector activity of both CD4^{+2,4} and CD8^{+3,5,13,14} T cells, can prevent the development of autoimmune disease¹⁵⁻¹⁷, and have been shown to play a role in both tumour immunity^{18,19} and transplantation^{13,20-24}. In vivo, but not in vitro, regulatory activity can be dependent on IL-10²⁵, TGF-β²⁶, and CTLA-4^{26,27}. In vitro studies with mouse cells have demonstrated that, although these regulatory populations require activation via their T cell receptors in order to regulate, once activated they can inhibit responses in an antigen non-specific manner, the process of ‘bystander regulation’^{2,4,6}. However, to date, the capacity of pre-activated T-reg to regulate non-specifically in vivo has not been tested.

The presence of T-reg with the capacity to suppress allograft rejection has been demonstrated in rodents with long term surviving cardiac^{13,20,21} and pancreatic islet^{22,23} allografts. It has previously been shown that pre-treatment of mice with donor-specific blood transfusion under the cover of anti-CD4 antibody allows the acceptance of fully allogeneic cardiac grafts²⁸. Using an adoptive transfer system it has been shown that pre-treatment of CBA (H2^{k}) mice with transfusion of blood from B10 (H2^{b}) mice under the cover of the anti-CD4 antibody YTS177 generates CD25⁺CD4⁺ cells that prevent rejection of donor-type skin allografts mediated by CD45RB^{high}CD4⁺ effector cells. Significantly, equal numbers of CD25⁻CD4⁺ cells from pre-treated animals or of CD25⁺CD4⁺ cells from naive mice or from mice pre-treated with antibody or transfusion alone were unable to regulate in this manner, demonstrating that these regulatory T cells (T-reg) arise entirely as a consequence of the full pre-treatment protocol²⁴. In common with naturally occurring CD25⁺CD4⁺ T-reg, regulation by these alloantigen-induced cells is dependent on IL-10 and CTLA-4.

These protocols, in which recipient mice are pre-treated before transplant with infusion of blood or bone marrow cells, expressing one or more histocompatibility antigens from the eventual transplant donor, in combination with anti-CD4 antibody, lead to the indefinite survival of heart allografts. It has recently been shown that this pre-treatment leads to the generation of regulatory cells prior to transplant which afford the graft protection from the outset. A pilot clinical study using a protocol based upon these data was performed at the Oxford Transplant Centre. The protocol was found to be safe and evidence supporting the generation of cells with regulatory activity was obtained.

However, there are several problems associated with the clinical use of protocols involving pre-treatment of patients with antigen prior to transplantation. Firstly there is a risk of transmission of blood-borne pathogens (for example HIV, hepatitis, BSE) when blood or cells are used in pre-treatment protocols. These risks must be taken into account and may preclude widespread use of such a protocol. A second major limiting factor in the clinical use of such protocols is the fact that, with the exception of live donor transplantation, neither the timing of the procedure nor the identity of the donor is known in advance. Thus it is impossible for the patient to undergo treatment with the relevant histocompatibility antigens from the eventual transplant donor, in combination with anti-CD4 antibody, in advance of the transplant itself.

An object of the present invention is to harness the potential of regulatory T cells in the suppression of transplant rejection and, in particular, to provide a method for the suppression of transplant rejection in an animal in which the disadvantages referred to above are alleviated or eliminated.

### SUMMARY OF THE INVENTION

According to one aspect, the present invention provides a method of suppressing rejection of an organ or tissue transplant in an animal comprising the following steps:
(a) administering to the animal an antibody directed at a cell surface antigen selected from the group consisting of CD4, CD8, CD154, LFA-1, CD80, CD86 and ICAM-1, and a non-cellular protein antigen to generate a population of regulatory T-lymphocytes;
(b) reactivating said population of regulatory T-lymphocytes by further administration to the animal of the non-cellular protein antigen; and
(c) transplanting said organ or tissue whilst said population of regulatory T-lymphocytes is activated.

Optionally, the method further comprises administering to the animal a population of regulatory T-lymphocytes produced according to an cc vivo method comprising culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen.

According to another aspect, the present invention provides the use of an antibody directed at a cell surface antigen selected from the group consisting of CD4, CD8, CD154, LFA-1, CD80, CD86 and ICAM-1, for the manufacture of a medicament for the suppression of rejection of an organ or tissue transplant in an animal by a method 5 which comprises administering the antibody to the animal together with a non-cellular protein antigen to generate in the animal a population of regulatory T-lymphocytes; reactivating said population of regulatory T-lymphocytes by further administration to the animal of the non-cellular protein antigen; and transplanting said organ or tissue whilst said population of regulatory T-lymphocytes is activated.

According to a further aspect, the present invention provides the use of a noncellular protein antigen for the manufacture of a medicament for the suppression of rejection of an organ or tissue transplant in an animal by a method which comprises administering an antibody directed at a cell surface antigen selected from the group consisting of CD4, CD8, CD154, LFA-1, CD80, CD86 and ICAM-1, to the animal together with the non-cellular protein antigen to generate in the animal a population of regulatory T-lymphocytes; reactivating said population of regulatory T-lymphocytes by further administration to the animal of the non-cellular protein antigen; and transplanting said organ or tissue whilst said population of regulatory T-lymphocytes is activated.

The invention further provides a method of treating a condition in an animal mediated by an immune response which comprises administering to said animal an antibody directed at a cell surface antigen selected from the group consisting of CD4, CD8, CD154, LFA-1, CD80, CD86 and ICAM-1, and a non-cellular protein antigen to generate a population of regulatory T-lymphocytes which are then re-activated by subsequent administration of the original non-cellular antigen.

Optionally, the method further comprises administering to the animal a population of regulatory T-lymphocytes produced according to an ex vivo method comprising culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen.

Another aspect of the invention provides the use of an antibody directed at a cell surface antigen selected from the group consisting of CD4, CD8, CD154, LFA-1, CD80, CD86 and ICAM-1, for the manufacture of a medicament for the treatment of a condition in an animal mediated by an immune response by a method which comprises administering the antibody to the animal together with a non-cellular protein antigen to generate in the animal a population of regulatory T-lymphocytes which are then re-activated by subsequent administration of the original non-cellular antigen.

A further aspect of the invention provides use of a non-cellular protein antigen for the manufacture of a medicament for the treatment of a condition in an animal mediated by an immune response by a method which comprises administering the non-cellular protein antigen to the animal together with an antibody directed at a cell surface antigen selected from the group consisting of CD4, CD8, CD154, LFA-1, CD80, CD86 and ICAM-1, to generate in the animal a population of regulatory T-lymphocytes which are then re-activated by subsequent administration of the original non-cellular antigen.

The invention further provides an ex vivo method for generating a population of regulatory T lymphocytes comprising culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen.

Another aspect of the invention relates to a method of suppressing rejection of an organ or tissue transplant in a recipient animal comprising the following steps:
(a) taking a sample of T cells from the recipient animal;
(b) taking a sample of alloantigen from a donor animal, said donor animal being the source of the organ or tissue being transplanted;
(c) exposing said sample of T cells to said sample of alloantigen in the presence of an antibody directed at a cell surface antigen selected from the group consisting of CD4, CD8, CD154,. LFA-1, CD80, CD86 and ICAM-1 to generate a population of regulatory T lymphocytes;
(d) administering to the recipient animal said population of regulatory T-lymphocytes.

### BRIEF DESCRIPTION OF THE DRAWINGS

FIG. 1 shows that alloantigen-induced CD25⁺ CD4⁺ cells generated in vivo can regulate skin allograft rejection;

FIG. 2 shows that cells generated by culturing CD4⁺ T cells with cells presenting alloantigen in vitro can regulate skin allograft rejection and that the regulatory activity can be further enriched by sorting CD62L⁺CD25+CD4⁺ cells generated by culturing CD4⁺ T cells with cells presenting alloantigen in vitro.

FIG. 3 shows that activated CD25⁺ CD4⁺ cells generated against unrelated antigen in vivo can regulate skin allograft rejection;

FIG. 4 shows that HGG re-challenge after 177/HGG pre-treatment in vivo leads to IFN-γ mRNA production;

FIG. 5 shows that when combined with a single dose of anti-CD8 antibody, the anti-CD4 HGG+re-boost protocol leads to prolonged cardiac allograft survival in primary recipients;

FIG. 6 shows proposed models for regulation of allograft rejection by regulatory cells.

### DETAILED DESCRIPTION OF THE INVENTION

According to one aspect of the present invention, regulatory T cells are generated by in vivo exposure to a non-cellular protein antigen in the presence of an antibody directed at a cell surface antigen selected from the group consisting of CD4, CD8, CD 154, LFA-1, CD80, CD86 and ICAM-1, preferably an anti-CD4 antibody. These T-reg are then re-activated by a second exposure to the non-cellular protein antigen prior to transplantation taking place. Once reactivated, the T-reg acquire the capacity to control the activity of graft destructive T cells so that transplant rejection can be suppressed or prevented provided that transplantation takes place whilst the T-reg are activated. Once transplantation has taken place, the protection provided by activation of the T-reg is sufficient for operational tolerance to the transplant to develop by other mechanisms. In recent years much progress has been made in defining the phenotypic and functional properties of T-reg. These cells can by enriched by sorting T cells that are CD4+ and express CD25, the α chain of the interleukin 2 receptor (IL-2R), and/or CD62L or other appropriate markers, and have been shown to express the transcription factor Foxp3. One characteristic that has been demonstrated by several groups in vitro is that these T-reg require activation via their T cell receptors in order to exert regulatory activity but that, once activated in this way, they are able to regulate in an antigen non-specific manner^{2,4,6}. According to the present invention, generation of T-reg by non-cellular protein antigen plus therapy with an antibody directed at a cell surface antigen selected from the group consisting of CD4, CD8, CD154, LFA-1, CD80, CD86 and ICAM-1, preferably anti-CD4 antibody therapy, followed by reactivation with the same protein allows them to regulate non-specifically to alloantigen in vivo.

The phenomenon of regulation in the specific setting of transplantation is of interest because active, self-sustaining regulation of rejection responses can provide a route to drug-independent long-term graft survival. As already noted, a major limiting factor in the clinical use of protocols involving pre-treatment of patients with antigen prior to transplantation is the fact that, with the exception of live donor transplantation, neither the timing of the procedure nor the identity of the donor is known in advance. Generation of regulatory cells by pre-treatment with a single graft alloantigen, which can prevent the rejection of fully allogeneic grafts, can widen the scope of such an approach and this phenomenon may contribute to the once well-recognised but poorly understood blood transfusion effect where pre-operative blood transfusion has a significant positive impact on graft outcome.³⁷⁻³⁹

However, even more attractive than the promotion of graft survival by pre-treatment with a limited subset of graft antigens would be the ability to achieve the same effect by the administration of antigens that are not necessarily expressed by the graft. The observation made in our laboratory that CD25⁺CD4⁺ cells from mice pre-treated with the anti-CD4 antibody YTS177 and third-party blood can prevent the rejection of unrelated B10 skin grafts suggests that this may indeed be feasible. The fact that regulation was only observed following re-challenge of the CD25⁺CD4⁺ cell donors with third party blood before transfer is entirely consistent with the observations of others that naturally-occurring T-reg are able to suppress in an antigen non-specific manner once activated.^{2,4,6}

According to the present invention, T-reg generated by administration of a non-cellular protein antigen such as human gamma globulin (HGG) combined with an antibody directed at a cell surface antigen selected from the group consisting of CD4, CD8, CD154, LFA-1, CD80, CD86 and ICAM-1, preferably anti-CD4 antibody, can prevent the rejection of grafts such as skin allografts. This is extremely attractive in view of the fact that the potential for the clinical transmission of infectious agents will limit the feasibility of the administration of human products such as blood. Clinical protocols are possible according to the present invention in which patients awaiting transplantation are given a well-defined, quality-controlled non-cellular antigen combined with immunotherapy with an antibody directed at a cell surface antigen selected from the group consisting of CD4, CD8, CD154, LFA-1, CD80, CD86 and ICAM-1, preferably anti-CD4 antibody immunotherapy, to generate T-reg. These T cells would be maintained by routine antigen re-challenge then re-activated immediately prior to transplantation. Under the correct circumstances these cells will be capable of regulating responses against the graft.

According to a further aspect of the invention, regulatory T cells are generated ex vivo by culturing T cells with an antibody directed at a cell surface antigen selected from the group consisting of CD4, CD8, CD154, LFA-1, CD80, CD86 and ICAM-1, in the presence of cells that present either alloantigen or a non-cellular protein antigen. The T cells may be taken from a recipient animal or patient. The resulting population of regulatory T cells can be introduced into the patient for use in prevention of transplant rejection or for treating autoimmune disease or graft-versus-host disease. In the case of use in the prevention of transplant rejection, the alloantigen may comprise cells taken from a donor animal or cells pulsed with antigen taken from a donor animal, wherein the donor animal may be the source of the transplanted organ or tissue.

These two approaches of overcoming transplant rejection can be used as alternatives or in combination. For example, regulatory T cells could be generated in vivo in a patient using the non-cellular protein antigen approach, and this could be followed post-transplant by treatment with regulatory T cells that have been generated ex vivo by using cells from the donor (whose identity would of course be known post-transplant) to activate T cells from the recipient patient in the presence of an antibody directed at a cell surface antigen selected from the group consisting of CD4, CD8, CD154, LFA-1, CD80, CD86 and ICAM-1.

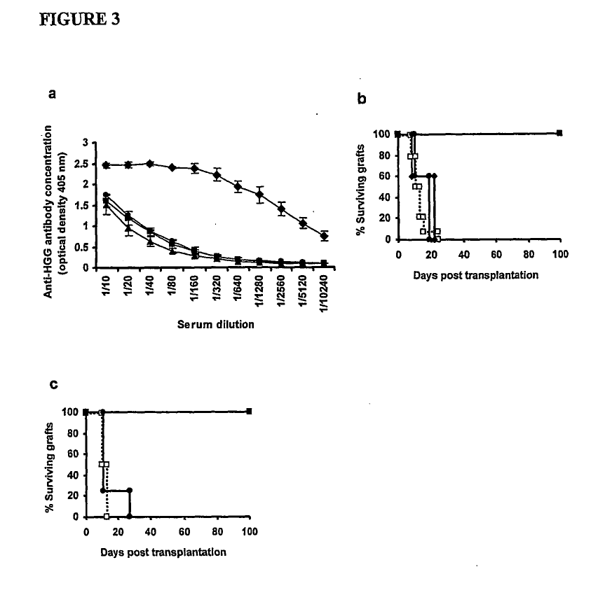

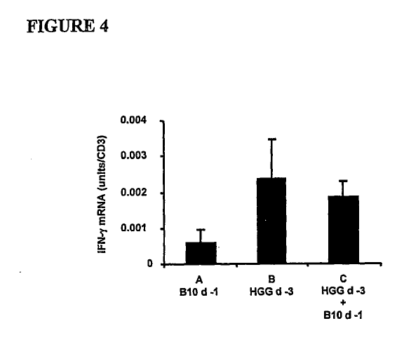

Our observations not only suggest potential strategies for immunotherapy but also shed some light on the mechanisms involved in regulation in vivo. T-regs generated by the 177/HGG−HGG re-challenge protocol have the ability to regulate rejection of B10 skin allografts suggesting that they might operate either by cross-reactivity or bystander regulation. Cross-reactivity seems the least likely of these two possibilities since it is presumed that these self-restricted CD25⁺ CD4⁺ T cells will have been generated in response to HGG peptides that are completely unrelated to alloantigen. The probability is that such cells act via bystander regulation in which activated T-reg regulate responses in a restricted local microenvironment, mediated by cytokines such as IL-10²⁴ (FIG. 3c) and IFN-γ (FIG. 4).

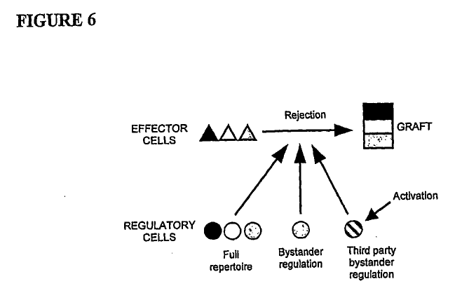

A proposed model to explain regulation in this system is summarised in FIG. 6. If T-reg populations are generated that are specific for each of the alloantigens expressed by the graft, rejection is prevented. However, since T-reg populations specific for a single donor antigen undergo activation by the graft, they are also able to overcome rejection through the process of bystander regulation. Prevention of graft rejection by T-reg that have been generated against non-graft antigens (which will not be activated by the graft) is possible only when the regulatory cells are first activated by deliberate antigen re-exposure. The duration of graft protection offered by these non-graft-specific T-reg is likely to be fairly short-lived since these cells are unlikely to remain activated for a significant length of time in the absence of specific antigen stimulation⁴⁰; however, it is probable that this period of protection allows operational tolerance to the graft to develop by other mechanisms⁴¹. The ability to generate T-reg populations by controlled exposure to defined antigens has important implications for clinical transplantation and may also have implications for autoimmune disease where attenuation of immune responses is also an important goal.

The present invention can be used in suppressing the rejection of allogeneic organs, tissues or cells of any type in an animal but is particularly applicable to the suppression of transplant rejection in humans.

The CD4 molecule expresses several different epitopes which in turn can lead to the production of different anti-CD4 antibodies after immunisation. All such anti-CD4 antibodies are capable of binding CD4 but they will have a range of properties based on affinity and isotype. For example, rat anti-mouse IgG2b antibodies deplete CD4+ T cells whereas broadly speaking IgG2a antibodies do not deplete. It has been shown that both depleting and non-depleting anti-CD4 antibodies can induce tolerance to an antigen and both can be used according to the present invention. However, non-depleting anti-CD4 antibodies may be preferred for use in clinical protocols since depletion of CD4+ T-cells may be difficult to control and long lasting.

The suitability of any particular anti-CD4 antibody for use according to the invention may be confirmed by the ability of the antibody to induce tolerance to a soluble non-cellular protein antigen such as HGG using the ELISA assay shown in FIG. 3a.

Other cell surface antigens may also be suitable targets for this type of approach including CD8, CD154 and LFA-1 on T cells and CD80, CD86 and ICAM-1 on antigen presenting cells. The ability of antibodies against such candidate molecules to induce tolerance to non-cellular protein antigens can also be determined by ELISA assays similar to that in FIG. 3a.

Many monoclonal antibodies have been described in the literature that would be suitable for use according to the present invention. Antibodies against the CD4 antigen and other cell surface molecules can be generated by standard methods involving the fusion of antibody secreting B cells with cell lines selected for their ability to confer in vitro immortality on the antibody secreting cells. Alternatively, DNA encoding monoclonal antibodies, antigen binding chains or domains can be cloned and expressed using standard methods of recombinant DNA technology. Recombinant antigen binding molecules can be manipulated to improve therapeutic properties such as specificity, affinity, half-life and lack of immunogenicity.

The use of antibodies generated in rodents or other non-human animals in therapy in man is limited by the reaction of the patient's immune system to these antibodies. However since anti-CD4 antibodies induce tolerance to themselves and the number of times that the anti-CD4 antibody needs to be administered when used according to the invention is limited, the anti-globulin response elicited by use of a rodent or other non-human antibody should be minimal. Accordingly, rodent (e.g. rat or mouse) or other non-human animal (e.g. horse) antibodies can be used according to the invention. However, for use in man it is preferred that the anti-CD4 antibody has been engineered to limit the anti-globulin response. Examples of antibodies engineered in this way are chimeric antibodies (where the constant regions of a non-human antibody are replaced by human constant regions) and humanised antibodies where the antibody is engineered to appear human to the immune system of the recipient. Examples of humanised antibodies are CDR-grafted antibodies where as well as replacing the constant regions of a non-human antibody with a human constant region, the framework regions of the variable regions are also replaced by human variable regions. Production of a humanised anti-CD4 antibody is described, for example, in WO-A-92 05274.

For the generation of a population of regulatory T cells in vivo the antibody may be administered intravenously by injection or infusion; intraperitoneal infusion is also possible. The antibody may be formulated for administration to humans in a standard manner, generally together with at least one physiologically acceptable carrier. The antibody will generally be formulated in solution in a physiologically acceptable carrier optionally with one or more other ingredients. Preferably the antibody is formulated in sterile isotonic buffered saline.

The non-cellular protein antigen is also generally administered parenterally, by intravenous infusion for the generation of a population of regulatory T cells in vivo. If a depot effect is required the non-cellular protein may be delivered by an intramuscular route. The antibody, preferably anti-CD4 antibody, is administered to the subject in a dose which is clinically effective to induce tolerance to an antigen in that subject. In any particular case, the precise dose will be at the discretion of the attendant physician but will generally be in the range 0.25 to 25 mg/kg. Generally from 1 to 5 doses but preferably 2 or 3 doses of the antibody, preferably anti-CD4 antibody, are given over a period of 2-5 days. The non-cellular protein antigen will be given concomitantly with the antibody to generate a population of regulatory T-lymphocytes which will then be expanded and/or maintained by repeated administration of the non-cellular antigen alone. The minimum number of doses of the antigen will be two but maintenance of the regulatory population may require 10 or more doses in total.

Suitable non-cellular protein antigens for use in accordance with the present invention should have the following characteristics:
(i) they should be immunogenic, i.e. must be a protein to which humans are not naturally tolerant;
(ii) the protein must be physiologically acceptable and non-toxic at the levels used.
(iii) administration of the tolerogenic protein with the immunomodulatory antibody should carry a minimal risk of sensitization. This may be assessed by established in vitro assays for the presence of circulating antibody.

Before any individual non-cellular soluble protein antigen is used in the method according to the present invention, it would need to have received regulatory approval for clinical use and proteins are preferred which have already received such approval. Examples of suitable non-cellular soluble protein antigens include human gamma globulin, equine gamma globulin and ovalbumin.

In a preferred embodiment the animal is treated with additional immunosuppression or adjunctive therapy to attenuate any immediate rejection response that occurs. The additional immunosuppression or adjunctive therapy may comprise administration of a sub-therapeutic dose of an immunosuppressive agent, preferably an agent used in a manner (time/dose) that does not block the function of the regulatory T cells, in the immediate post-operative period. Suitable immunosuppressive agents or adjunctive therapies include treatment with an anti-CD8 antibody or with rapamycin. The intention is that the combination of the antibody plus non-cellular protein antigen treatment with a sub-therapeutic dose of an immunosuppressive agent would lead to the prolonged survival of fully allogeneic cardiac allografts in fully immunocompetent recipients. A sub-therapeutic dose can be identified by reference to clinical studies identifying suitable therapeutic doses.

On the basis of surface phenotype, T cells can be sub-divided into CD4⁺ and CD8⁺ populations and both can play non-overlapping roles in graft rejection in intact animals. By definition the anti-CD4/HGG+HGG re-challenge protocol targets only CD4⁺ T cells. The use of an anti-CD8 monoclonal antibody (e.g. YTS 169) to target CD8 T cells, which in many situations are capable of mediating graft rejection independently of CD4 T cells, should therefore have an important influence on the efficacy of the anti-CD4/HGG+HGG re-challenge protocol.

The alloantigen used in the ex vivo method may be cells from a donor animal or cells pulsed with antigen. In the case of a living donor the cells could be isolated from peripheral blood and in the case of a cadaveric donor the cells could either be isolated from the spleen or from donor-peripheral blood. If the antigen of interest has not been identified, cells can be pulsed with a mixture of antigens obtained from pancreatic cells of the donor animal which is likely to include the antigen of interest.

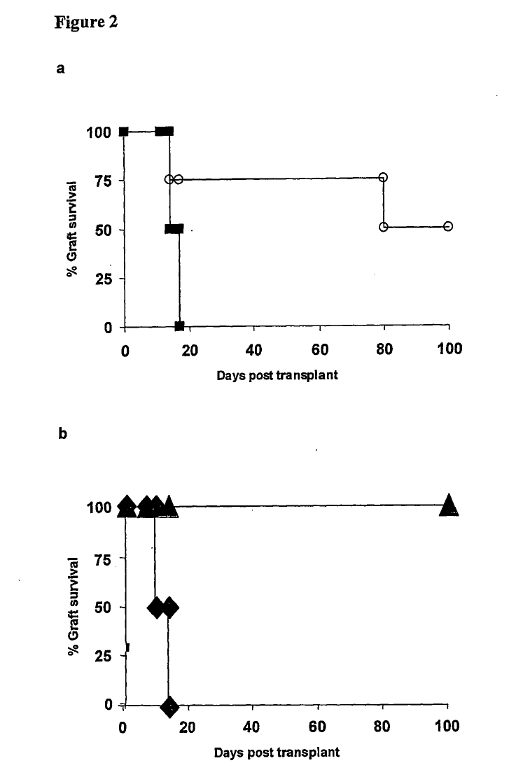

Regulatory T cells generated ex vivo by exposing CD4⁺ T cells in culture to cells presenting donor alloantigen or an unrelated protein antigen in the presence of an antibody directed at a cell surface antigen selected from the group consisting of CD4, CD8, CD154, LFA-1, CD80, CD86 and ICAM-1, preferably an anti-CD4 antibody, could be used either as an alternative or complementary therapeutic approach to prevent or treat transplant rejection. T cells generated in the cultures would then be administered intravenously to the animal either before or after transplantation (FIG. 2a). T cells with regulatory activity generated in the cultures can be further enriched by purifying cells express CD62L, i.e. CD62L⁺CD25⁺CD4⁺ T cells (FIG. 2b).

The present invention also provides a method of treating a condition in an animal mediated by an immune response which comprises administering to said animal an antibody directed at a cell surface antigen selected from the group consisting of CD4, CD8, CD154, LFA-1, CD80, CD86 and ICAM-1, preferably an anti-CD4 antibody, and a non-cellular protein antigen to generate a population of regulatory T-lymphocytes which are then re-activated by subsequent administration of the original non-cellular antigen.

The condition mediated by an immune response is generally an autoimmune condition. Examples of autoimmune conditions include rheumatoid arthritis, multiple sclerosis insulin-dependent diabetes melitus and inflammatory bowel disease. CD4+ T cells play a central role in autoimmunity and have the capacity to be both protective and pathogenic. Accumulating evidence suggests that autoimmunity probably results when normal regulatory functions of protective CD4+ T cells break down. Autoimnuune diseases can be treated to a certain extent by manipulation of CD4+ T cells. However, the effects may be only transient due to T cell turn-over and re-acquisition of T cell function. If such recovering T cells re-encounter auto-antigens that initiated the initial disease during on-going inflammation of the target tissue (for example the synovial joint in rheumatoid artritis, pancreatic β-cells in insulin-dependent diabetes) the T cells will become activated and autoimmune destruction will re-occur. It may be possible to re-establish a balance between pathogenic and protective T cells by transient therapy designed to disable/deplete activated T cells followed by the administration of a non-cellular protein antigen such as HGG combined with additional or adjunctive immunotherapy using for example anti-CD4 antibody. Regulatory T cells generated in this way would be reactivated by further administration of the non-cellular protein antigen and these might migrate to sites of inflammation along chemokine gradients and could arrive in the joints ready to suppress autoreactive T cells. Thus, a protocol such as this would involve firstly depletion or inactivation of auto-reactive cells, secondly administration of non-cellular protein antigen together with monoclonal antibody immunotherapy and thirdly, reactivation of putative regulatory cells by subsequent administration of repeated doses of the original non-cellular antigen.

Additionally, regulatory T cells generated ex vivo by exposing CD4⁺ T cells in culture to cells presenting donor alloantigen or an unrelated protein antigen in the presence of an antibody directed at a cell surface antigen selected from the group consisting of CD4, CD8, CD154, LFA-1, CD80, CD86 and ICAM-1, preferably an anti-CD4 antibody, could be used either as an alternative or complementary therapeutic approach to treat the condition mediated by an immune response. T cells generated in the cultures would be administered intravenously to the animal.

### EXAMPLES

The invention is based on and illustrated by the following experimental work. The experiments are described in general terms followed by more detailed description with reference to the Figures and lastly more detailed description of some of the methods.

Experiment 1

Alloantigen-Induced T-Regs Control the Rejection of Donor-Specific Skin Grafts

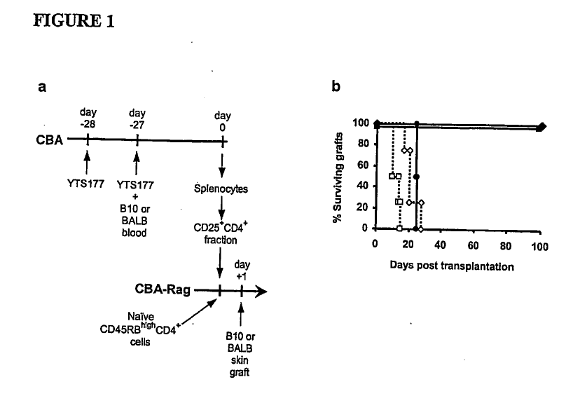

In order to provide a basis for answering questions about the antigen specificity of T-reg, CD25⁺CD4⁺ cells were isolated from CBA (H2) mice pre-treated with the anti-CD4 antibody YTS 177 together with blood from either B10 (H₂b) or BALB H₂^{d}) mice and transferred them into syngeneic immunodeficient CBA-Rag^{-/-} mice together with CD45RB^{high}CD4⁺ effector cells. One day later these recipients were transplanted with skin grafts either matched to the blood transfusion donor or from a third party strain (FIG. 1a). Animals reconstituted with effector cells alone acutely rejected both B10 and BALB skin allogras, and as shown previously²⁴ CD25⁺CD4⁺ cells isolated from mice pre-treated with YTS177 and B10 blood prevented the rejection of donor-specific B10 allografts (FIG. 1b). This phenomenon was not restricted to a single donor/recipient strain combination, as CD25⁺CD4⁺ cells from mice pre-treated with YTS177 and BALB blood prevented the rejection of BALB skin grafts. However, CD25⁺CD4⁺ cells from animals pre-treated with YTS177 and BALB blood were unable to prevent the acute rejection of B10 skin grafts, clearly demonstrating that regulatory cell function under these conditions in vivo is alloantigen-specific (FIG. 1b).

Experiment 2

Regulatory T Cells Generated In Vitro by Pre-Culturing CD4⁺ T cells with Anti-CD4 Monoclonal Antibody (mAb) Can Control the Rejection of Donor-Specific Skin Grafts

CD4⁺ T cells from naive CBA mice were cultured with irradiated antigen presenting cells from C57Bl/10 mice in the presence of 5 μg/ml of 177 YTS anti-CD4 antibody.

After 8 days in culture total CD4⁺ or CD62L⁺CD25⁺CD4⁺CBA T cells pre-cultured with anti-CD4 mAb were transferred into syngeneic immunodeficient CBA-Rag^{-/-} mice together with CD45RB^{high}CD4⁺ effector cells. One day later these recipients were transplanted with skin grafts matched to the alloantigen used for the in vitro culture. Animals reconstituted with effector cells alone acutely rejected both B10 skin allografts, while cells isolated from the in vitro cultures prevented the rejection of donor-specific B10 allografts (FIG. 2a). The population of regulatory T cells generated in vitro could be further enriched by purifying cells CD62L+ CD25+CD4+ cells (FIG. 2b ).

Experiment 3

Activated T-Regs Generated Against Unrelated Antigen Can Regulate Skin Allograft Rejection

Generating T-reg specific for a noncellular protein antigen could offer a potential tolerance induction strategy in man if such cells could regulate responses to alloantigens in vivo once re-activated. It has previously been demonstrated that tolerance to soluble antigens such as albumin³⁴ or human gamma globulin (HGG)^{35,36} can be achieved by intravenous administration to mice under the cover of anti-CD4 antibody. We therefore chose HGG as a candidate for the tolerising antigen for these experiments. First, in order to determine whether induction of tolerance to HGG could be replicated in our hands we administered YTS177 and HGG and then measured serum anti-HGG antibody concentrations by ELISA (FIG. 3a). Positive control mice that received a priming protocol (where HGG was given without YTS177) produced high levels of anti-HGG antibody whereas mice given HGG under the cover of YTS177 gave low antibody titers identical to those from unprimed naive mice, indicating tolerance to HGG. Importantly, mice pre-treated according to the tolerising plus re-activation protocol (antigen under the cover of YTS177 followed by a second dose of antigen the day prior to analysis) produced background levels of anti-HGG antibody and were therefore judged to be tolerant to HGG.

Having confirmed that tolerance to HGG can be induced in this way FIG. 2a), the next question was whether T-regs cells generated by pre-treatment with HGG under the cover of YTS 177 and specifically reactivated with HGG could regulate the rejection of B10 skin allografts (FIG. 3b). Animals reconstituted with CD₄5RB^{high}CD4⁺ effector cells alone all acutely rejected their grafts but, in contrast, all mice that received co-transfer of CD25⁺CD4⁺ cells from animals pre-treated with YTS177 and HGG and then given a second dose of HGG the day prior to cell isolation (to re-activate the regulatory population) accepted their B10 skin allografts for >100 days. As important controls, we also tested the regulatory capacity of CD25⁺CD4⁺ cells from animals pre-treated with YTS 177 and HGG but without the second (re-activating) dose of HGG, and from animals pre-treated with HGG in the absence of YTS 177 followed by a second dose of HGG prior to cell isolation. In both these groups all B10 skin allografts were rejected acutely. In common with regulation by alloantigen-induced CD25⁺CD4⁺Treg²⁴, regulation by CD25⁺CD4⁺ cells from mice pre-treated with YTS 177 and HGG and then reactivated by a second dose of HGG was EL-10-dependent in that blockade of the IL-10 axis abrogated regulation and led to acute rejection (FIG. 3c).

Experiment 4

Re-Challenge of Mice with HGG Following 177/HGG Pre-Treatment Leads to IFN-γ mRNA Production

Previous work on the YTS 177/DST tolerance induction model described above (FIG. 1) has demonstrated that CD25⁺CD4⁺ cells isolated from tolerised mice show a rapid but transient up-regulation of IFN-γ mRNA following re-exposure to the original tolerising antigen. This IFN-γ signal was only evident in mice given the combined 177/DST protocol suggesting a possible role for IFN-γ in the induction of tolerance in this model. In order determine whether a similar IFN-γ signature was a feature of the 177/HGG model shown in FIG. 3, CBA mice were pre-treated with the 177/HGG protocol and then re-boosted with HGG and/or given a B10 blood transfusion (to mimic exposure to alloantigen normally in the form of a skin graft) as described (FIG. 4). Mice given the non-effective 177/HGG pre-treatment without an HGG re-boost (FIG. 3b) showed a low level of IFN-γ mRNA expression (column A). However, cells from mice given the tolerising protocol followed by an HGG re-boost showed a three-fold increase in IFN-γ message upon exposure to alloantigen (column C). Most significantly, this increase was also seen in tolerised mice given the HGG re-boost without alloantigen (column B) demonstrating that in the adoptive transfer system shown above (FIG. 3b) CD25⁺CD4⁺ Treg would already be expressing elevated levels of IFN-γ mRNA at the time of skin grafting.

Experiment 5

When Combined with Adjunctive ant-CD8 Antibody Therapy the 177/HGG+HGG Re-Boost Protocol Leads to Long-Term Allograft Survival in Immunocompetent Recipients.

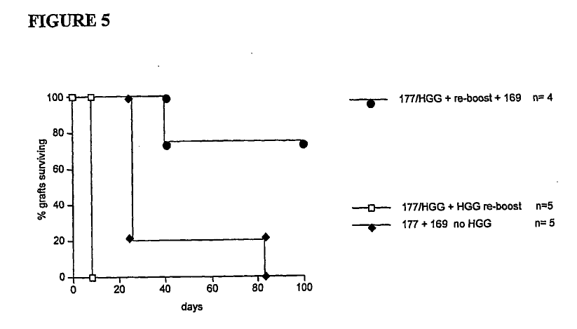

The 177/HGG+HGG re-boost protocol generates CD25⁺CD4⁺ T cells that on adoptive transfer to reconstituted immunodeficient mice prevent skin graft rejection (FIG. 3). In order to determine whether the same induction protocol could protect allografts in primary immunocompetent recipients, CBA (H-2^{k}) mice were pretreated with the 177/HGG+HGG re-boost protocol then transplanted with C57BL/10 (H-2^{b}) cardiac allografts on day 0. As shown in FIG. 5, the 177/HGG+HGG re-boost protocol had no effect on graft outcome in that all mice rejected their grafts at control rates (n=5, median graft survival time, MST, 8 days). It is well established that both CD4⁺ and CD8⁺ T cells can contribute to allograft rejection and although CD25⁺CD4⁺ regulatory T cells may be capable of regulating CD8 T cell responses it seemed likely that the lack of effect of the 177/HGG+HGG in normal mice could be due to an inability of the T-reg population in this situation to control alloreactive CD8⁺ T cells. To test this possibility CBA mice were pretreated with the 177/HGG+HGG re-boost protocol and in addition received anti-CD8 at the time of transplant. Three out of four mice in this group accepted their C57BL/10 cardiac allografts for greater than 100 days (FIG. 5). Control mice given anti-CD4 antibody (d-28, -27) plus anti-CD8 antibody (day 0) without HGG rejected their cardiac allografts with an MST of 26 days (n=5).

Detailed Description of the Figures

FIG. 1: Alloantigen-Induced CD25⁺CD4⁺ Cells Can Regulate Skin Allograft Rejection

a, Pre-treatment and adoptive transfer protocol. CBA mice were pre-treated with YTS177 on days -28 and -27 together with allogeneic (1B10 or BALB) blood transfusion on day -27). On day 0, CD25⁺CD4⁺ cells from the spleens of these animals were adoptively transferred into CBA-Rag^{-/-} recipients together with CD45RB^{high}CD4⁺ cells from naive animals, and the following day a B10 or BALB skin allograft was performed. b, Effect of CD25⁺CD4⁺ cells on CD45RB^{high}CD4⁺-mediated skin allograft rejection. All mice were reconstituted with CD45RB^{high}CD4⁺ cells with or without different CD25⁺CD4⁺ populations and received the following skin grafts: □: B10 graft (group i: MST=20 days, n=4); ⋄: BALB graft (group ii: MST=12 days, n=4); ▪: CD25⁺CD4⁺ cells from YTS177/B10 blood pre-treated mice+B10 graft (group iii: MST>100 days, n=4; P<0.05 compared to group i); ♦: CD25⁺CD4⁺ cells from YTS177/BALB blood pre-treated mice+BALB graft (group iv: MST>100 days, n=4; P<0.05 compared to group ii); ●: CD25⁺CD4⁺ cells from YTS177/BALB blood pre-treated mice+B10 graft (group v: MST 25 days, n=4; P<0.05 compared to groups iii and iv.

FIG. 2: T-Cells Generated In Vitro by Co-Culturing CD4+ T Cells with Cells Presenting Donor Alloantigens Can Regulate Skin Allograft Rejection

a, Effect of cells generated in vitro by co-culturing CD4⁺ T cells with cells presenting donor alloantigens and anti-CD4 monoclonal antibody on CD45RB^{high}CD4⁺-mediated skin allograft rejection. Mice were reconstituted with 1×10⁵ CD45RB^{high}CD4⁺ cells alone and received B10 skin grafts the following day ▪ (group i: MST=20 days, n=4); or were reconstituted with 1×10⁵ CD₄₅RB^{high}CD4⁺ cells and 2×10⁵ cells generated in vitro by co-culturing CD4⁺ T cells with cells presenting B10 alloantigens and 5 μg/ml anti-CD4 monoclonal antibody for 8 days and received B10 skin grafts o (group ii: MST>100 days, n=4; P<0.05 compared to group i); b, Effect of CD62L⁺CD25⁺CD4⁺ cells generated in vitro by co-culturing CD4⁺ T cells with cells presenting donor alloantigens, on CD45RB^{high}CD4⁺-mediated skin allograft rejection. Mice were reconstituted with 1×10⁵ CD45RB^{high}CD4⁺ cells alone and received B10 skin grafts the following day ♦ (group i: MST=13 days, n=6); or were reconstituted with 1×10⁵ CD₄₅RB^{high}CD4⁺ cells and 2×10⁵ CD62L⁺CD25⁺CD4⁺ cells generated in vitro by co-culturing CD4⁺ T cells with cells presenting B10 alloantigens and 5 μg/ml anti-CD4 monoclonal antibody for 8 days and received B10 skin grafts ▴ (group ii: MST>100 days, n=4; P<0.05 compared to group i)

FIG. 3: Activated CD25⁺CD4⁺ Cells Generated Against Unrelated Antigen Can Regulate Skin Allograft Rejection

a, Induction of tolerance to HGG. CBA mice were pre-treated as follows and serum anti-HGG antibody titer was measured by ELISA: ▴: naïve; ●: tolerizing protocol—YTS177 on days -42, -41, and -40, and HGG on days -41, -14, and -7; ♦: priming protocol—YTS177 on days -42, -41, and -40, and HGG on days -14 and -7; ▪: protocol used for in vivo adoptive transfer—YTS177 on days -28 and -27 and HGG on days -27 and -1. n=2 in each group, results are presented as mean ± standard deviation. b, Effect of different CD25⁺CD4⁺ populations on skin allograft rejection. CBA-Rag^{-/-} mice were reconstituted with CD45RB^{high}CD4⁺ cells together with the following CD25⁺CD4⁺ populations from pre-treated CBA mice and then received a B10 skin allograft: □: no CD25⁺CD4⁺ cells (group i: MST=11.5 days, n=14); ▪: CD25⁺CD4⁺ cells from mice pre-treated with YTS177 on days -28 and -27 and with HGG on days -27 and -1 (group ii: MST>100 days, n=5; P<0.05 compared to group i); ●: CD25⁺CD4⁺ cells from mice pre-treated with HGG only on days -27 and -1 (group iii: MST 20 days, n=5; P=0.21 compared to group i); ♦: CD25⁺CD4⁺ cells from mice pre-treated with YTS177 only on days -28 and -27 and HGG on day -27 (group iv: MST 21 days, n=5; P=0.21 compared to group i). c, Effect of IL-10R blockade on regulation of B10 skin allograft rejection by CD25⁺CD4⁺ cells from mice pre-treated with YTS177 on days -28 and -27 and HGG on days -27 and -1. CBA-Rag^{-/-} mice were reconstituted with CD45RB^{high}CD4⁺ cells with or without CD25⁺CD4⁺ cells from pre-treated animals and antibody therapy. □: CD45RB^{high}CD4⁺ cells only (group i: MST 11.5 days, n=4); ●: CD45RB^{high}CD4⁺ and CD25⁺CD4⁺ cells and anti-IL10R blockade with 1B1.2 (group ii: MST 10 days, n=4; P=0.81 compared to group i); ▪: CD45RB^{high}CD4⁺ and CD25⁺CD4⁺ cells and isotype control antibody GL113 (group iii: MST>50 days, n=4; P<0.05 compared to groups i and ii).

FIG. 4: HGG Re-Challenge After 177/HGG Pre-Treatment Leads to IFN-γ mRNA Production

CBA mice were pre-treated with YTS177 on days -28 and -27 and HGG on day -27. Group a then received B10 blood on day -1, group B HGG on day -3, and group C HOG on day-3 plus B10 blood on day -1. On day O CD25⁺CD4⁺ splenocytes were purified and IFN-γ mRNA measured by RT-PCR. Results show the mean of three independent experiments.

FIG. 5: Pretreatment with the Anti-CD4/HGG+HGG Protocol Combined with Peri-Transplant Anti-CD8 Antibody Leads to Long-Term Allograft Survival in Immunocompetent Primary Recipients

CBA (H-2^{k}) mice were given YTS 177 (8 mg/kg) day -28, -27 and HGG (20 mg/kg) day -27 and day -1 plus YTS 169 (8 mg/kg) day 0, (●, n=4). Control mice received YTS 177 (8 mg/kg) day -28, -27 and HGG (20 mg/kg) day -27 and day -1 without anti-CD8 antibody (□, n=5) or YTS 177 (8 mg/kg) day -28, -27 plus YTS 169 (8 mg/kg) day 1 without HGG (♦, n=5). All mice were transplanted with C57BL/10 (H-2^{b}) hearts on day 0.

FIG. 6: Proposed Models for Regulation of Allograft Rejection by Regulatory Cells

Rejection may be overcome by the generation of regulatory populations specific for all of the alloantigens expressed on a graft (full repertoire). Alternatively regulatory populations may be generated against a single graft antigen; these regulatory cells undergo activation by the graft and then suppress responses against other graft antigens (bystander regulation). Finally regulatory cells generated against third party or even completely unrelated antigen can suppress graft rejection by bystander regulation provided that they are first activated before their functional activity is tested.

Methods

Mice

CBA.Ca (CBA, H2k), C57BL/10 (B10, H2^{b}), BALB/c (BALB, H2^{d}), CBK (H2^{k}+K^{b}, kindly provided by Dr. A. L. Mellor, Medical College, Augusta, Ga., U.S.A.), and CBA-Rag 1^{-/-} (CBA-Rag^{-/-}, H2^{k}, kindly provided by Dr. D. Kioussis, Division of Molecular Immunology, National Institute for Medical Research, Mill Hill, London, U.K.) were obtained from and housed in the Biomedical Services Unit, John Radcliffe Hospital (Oxford, U.K.). Sex-matched mice between 6 and 12 weeks of age the time of first experimental procedure were used in all experiments.

Reagents and Monoclonal Antibodies

The following antibodies were used for cell purification, flow cytometry, and in vivo administration. The hybridoma TIB120 (anti-MHC class II) was obtained from American Type Culture Collection (ATCC), Manassas, Va., U.S.A.; YTS169 (anti-CD8) and YTS177.9 (anti-CD4)³⁶ were kindly provided by Professor H. Waldmann (Sir William Dunn School of Pathology, Oxford, U.K.). RM4-5 (anti-CD4)-CyC, 16A (anti-CD45RB)-PE, 7D4 (anti-CD25)-biotin, and streptavidin-PE were purchased from Pharmingen (San Diego, Calif., U.S.A.). 1B1.2, a blocking rat IgG1 antibody reactive with mouse IL-10R (ref. 42). GL113, a rat IgG1 isotype control antibody reactive with β-galactosidase⁴³.

Human gamma globulin (HGG) was purchased from Sigma-Aldrich (St. Louis, Mo., U.S.A.) and was heat aggregated at 63° C. for 25 minutes and then incubated overnight on ice prior to use.

In Vivo Pre-Treatment Protocol

Adult CBA mice received 200 μg of the anti-CD4 mAb YTS 177 intravenously on days -28 and -27. On day -27 they also received 250 μl of allogeneic (1310 or BALB) blood or 500 μg of HGG intravenously. In some experiments a further dose of allogeneic blood or HGG was administered on day -1. Spleens were harvested on day 0 for cell isolation of CD25⁺CD4⁺ cells.

In Vitro Protocol

Purified CD4⁺ T cells from naive CBA mice were cultured at 2×10⁵ cells per well in the presence of 5 μg/ml of YTS 177 anti-CD4 monoclonal antibody together with 5×10⁵ irradiated (3600 rad) allogeneic total splenocytes per well from B10 mice. Culture medium was composed of RPMI 1640 supplemented with 10% FCS (both P.A.A. Laboratories GmBH, Linz, Austria), 2 mM L-Glutamine, 0.5 mM 2-mercaptoethanol (Sigma, St Louis, Mo., U.S.A.), and 100 units/ml each penicillin, streptomycin and kanamycin. All cultures were set in U shaped 96 well plates (Corning Costar, Cambridge, Mass.). Cells were kept in culture for 8 days at 37° C., 5% CO₂ and then harvested.

Cell Purification

CD4⁺ (in vitro cultures) and CD45RB^{high}CD4⁺ (in vivo adoptive transfer experiments) T cells were isolated from lymph nodes and spleens of naive CBA mice, and CD25⁺CD4⁺ T cells were obtained from spleens of animals pre-treated with YTS177 and allogeneic blood or HGG. CD25⁺CD4⁺ and CD62L⁺CD25⁺CD4⁺ T cells were obtained from in vitro co-cultures of CD4⁺ T cells with cells presenting donor alloantigens and anti-CD4 monoclonal antibody (YTS177). Populations were purified by negative selection using magnetic beads followed by FACS sorting as previously described.²⁴ On re-analysis, all populations were >95% pure.

Cell Adoptive Transfer and Skin Transplantation

CBA-Rag^{-/-} mice were reconstituted intravenously with 10⁵ CD45RB^{high}CD4⁺ cells with or without 2×10⁵ CD25⁺CD4⁺ cells. The following day full thickness B10 or BALB tail skin allografts were transplanted onto graft beds prepared on the flanks of the reconstituted mice. Where appropriate anti-IL10R antibody (or isotype control) was administered intraperitoneally at a dose of 1 mg at the time of cell adoptive transfer and then 0.5 mg per week thereafter for 6 weeks or until graft rejection occurred. Allografts were monitored and graft survival between groups was compared using the log rank test⁴⁴ using software developed and kindly provided by Dr. S. Cobbold, Sir William Dunn School of Pathology, Oxford, U.K.

Histological Examination

Skin grafts were fixed in buffered 10% formalin. 6 μm paraffin-embedded sections were cut and stained with hematoxylin and eosin.

Serum Anti-HGG Antibody ELISA

Serum concentration of anti-HGG antibodies was measured by ELISA using a modification of standard methods. Plate-bound HGG was used to capture serum anti-HGG antibody which was then revealed and quantified using horseradish peroxidase-conjugated rabbit anti-mouse IgG and IgM (Jackson ImmunoResearch Laboratories, West Grove, Pa., U.S.A.) followed by ABTS (2,2′-azino-bis(2-ethyl-benzathiazoline-6-sulfonic) acid)⁴⁵. Absorbance at 405 nm was read and results are presented as the mean of duplicate wells ± standard deviation.

Heart Transplantation

C57BL/10 cardiac allografts were transplanted into the abdomen of CBA recipients as described²⁸. Briefly, the donor aorta and pulmonary artery were anastomosed end-to side to the recipient descending aorta and inferior vena cava respectively. Graft function was followed by abdominal palpation and graft function at 100 days post transplant confirmed by laparotomy and direct visual inspection.

Abbreviations:

| | |
|---|---|
| HGG | Human gamma globulin |
| mAb | Monoclonal antibody |
| MST | Median survival time |
| RT-PCR | Real time polymerase chain reaction |
| Treg | Regulatory T cell |

### REFERENCES

1. Gershon, R. K. & Kondo, K. Infectious immunological tolerance. Immunology 21, 903-914 (1971).
2. Takahashi, T. et al Immunologic self-tolerance maintained by CD25+CD4+ naturally anergic and suppressive T cells: induction of autoimmune disease by breaking their anergic/suppressive state. Int. Immunol. 10, 1969-1980 (1998).
3. Gao, Q., Rouse, T. M., Kazmerzak, K. & Field, E. H. CD4+CD25+ cells regulate CD8 cell anergy in neonatal tolerant mice. Transplantation 68, 1891-1897 (1999).
4. Thornton, A. M. & Shevach, E. M. Suppressor effector function of CD4+CD25+ immunoregulatory T cells is antigen nonspecific. J. Immunol. 164, 183-190 (2000).
5. Piccirillo, C. A. & Shevach, E. M. Cutting edge: control of CD8+ T cell activation by CD4+CD25+ immunoregulatory cells. J. Immunol. 167, 1137-1140 (2001).
6. Chai, J.-G. et al. CD4+CD25+ T cells as immunoregulatory T cells in vitro. Eur. J. Immunol. 32, 2365-2375 (2002).
7. Baecher-Allan, C., Brown, J. A., Freeman, G. J. & Hafler, D. A. CD4+CD25 high regulatory cells in human peripheral blood. J. Immunol 167, 1245-1253 (2001).
8. Jonuleit, H. et al. Identification and functional characterization of human CD4+CD25+ T cells with regulatory properties isolated from peripheral blood. J. Exp. Med. 193, 1285-1294 (2001).
9. Dieckmann, D., Plottner, H., Berchtold, S., Berger, T. & Schuler, G. Ex vivo isolation and characterization of CD4+CD25+ T cells with regulatory properties from human blood. J. Exp. Med. 193, 1303-1310 (2001).
10. Levings, M. K., Sangregorio, R. & Roncarolo, M.-G. Human CD25+CD4+ T regulatory cells suppress naive and memory T cell proliferation and can be expanded in vitro without loss of function. J. Exp. Med. 193, 1295-1302 (2001).
11. Stephens, L. A., Mottet, C., Mason, D. & Powrie, F. Human CD4+CD25+ thyrocytes and peripheral T cells have immune suppressive activity in vitro. Eur. J. Immunol. 31, 1247-1254 (2001).
12. Taams, L. S. et al. Human anergic/suppressive CD4+CD25+ T cells: a highly differentiated and apoptosis-prone population. Eur. J. Immunol. 31, 1122-1131 (2001).
13. Van Mauik, A., Herber, M., Wood, K. J. & Jones, N. D. Cutting edge: CD4+CD25+ alloantigen-specific immunoregulatory cells that can prevent CD8+ T cell-mediated graft rejection: implications for anti-CD154 immunotherapy. J. Immunol. 169, 5401-5404 (2002).
14. Lin, C.-Y., Graca, L., Cobbold, S. P. & Waldmann, H. Dominant transplantation tolerance impairs CD8+ T cell function but not expansion. Nat. Immunol. 3, 1208-1213 (2002).
15. Sakaguchi, S., Sakaguchi, N., Asano, M., Itoh, M. & Toda, M. Immunologic self-tolerance maintained by activated T cells expressing IL-2 receptor alpha-chains (CD25). Breakdown of a single mechanism of self-tolerance causes various autoimmune diseases. J. Immunol. 155, 1151-1164 (1995).
16. Taguchi, O. & Takahashi, T. Administration of anti-interleukin-2 receptor alpha antibody in vivo induces localized autoimmune disease. Eur. J. Immunol. 26, 1608-1612 (1996).
17. Suri-Payer, E., Amar, A. Z., Thornton, A. M. & Shevach, E. M. CD4+CD25+ T cells inhibit both the induction and effector function of autoreactive T cells and represent a unique lineage of immunoregulatory cells. J. Immunol. 160, 1212-1218 (1998).
18. Onizuka, S. et al. Tumor rejection by in vivo administration of anti-CD25 (interleukin-2 receptor alpha) monoclonal antibody. Cancer Res. 59, 3128-3133 (1999).
19. Golgher, D., Jones, E., Powrie, F., Elliott, T. & Gallimore, A. Depletion of CD25+ regulatory cells uncovers immune responses to shared murine tumor rejection antigens. Eur. J. Immunol. 32, 3267-3275 (2002).
20. Hall, B. M., Pearce, N. W., Gurley, K. E. & Dorsch, S. E. Specific unresponsiveness in rats with prolonged cardiac allograft survival after treatment with cyclosporine. m. Further characterization of the CD4+ suppressor cell and its mechanisms of action. J. Exp. Med. 171, 141-157 (1990).
21. Hara, M. et al. IL-10 is required for regulatory T cells to mediate tolerance to alloantigens in vivo. J. Immunol. 166, 3789-3796 (2001).
22. Sanchez-Fueyo, A., Weber, M., Domenig, C., Stron, T. B. & Zheng, X. X. Tracking the immunoregulatory mechanisms active during allograft tolerance. J. Immunol. 168, 2274-2281 (2002).
23. Gregori, S. et al. Regulatory T cells induced by 1 alpha,25-dihydroxyvitamin D3 and mycophenolate mofetil treatment mediate transplantation tolerance. J. Immunol. 167, 1945-1953 (2001).
24. Kingsley, C. I., Karim, M., Bushell, A. R. & Wood, K. J. CD25+CD4+ regulatory T cells prevent graft rejection: CTLA-4- and IL-10-dependent immunoregulation of alloresponses. J. Immunol. 168, 1080-1086 (2002).
25. Asseman, C., Mauze, S., Leach, M. W., Coffman, R. L. & Powrie, F. An essential role for interleukin 10 in the function of regulatory T cells that inhibit intestinal inflammation. J. Exp. Med. 190, 995-1004 (1999).
26. Read, S., Malmstrom, V. & Powrie, F. Cytotoxic T lymphocyte-associated antigen 4 plays an essential role in the function of CD25+CD4+ regulatory cells that control intestinal inflammation. J. Exp. Med. 192, 295-302 (2000).
27. Liu, Z. et al. B7 interactions with CD28 and CTLA-4 control tolerance or induction of mucosal inflammation in chronic experimental colitis. J. Immunol. 167, 1830-1838 (2001).
28. Pearson, T. C., Madsen, J. C., Larsen, C. P., Morris, P. J. & Wood, K. J. Induction of transplantation tolerance in adults using donor antigen and anti-CD4 monoclonal antibody. Transplantation 54, 475-483 (1992).
29. Pescovitz, M. D. et al. Effect of class II antigen matching on renal allograft survival in miniature swine. J. Exp. Med. 160, 1495-1508 (1984).
30. Hutchinson, I. V., Barber, W. H. & Morris, P. J. Specific suppression of allograft rejection by trinitrophenyl (TNP)-induced suppressor cells in recipients treated with TNP-haptenated donor alloantigens. J. Exp. Med. 162, 1409-1420 (1985).
31. Madsen, J. C., Superina, R. A., Wood, K. J. & Morris, P. J. Immunological unresponsiveness induced by recipient cells transfected with donor MHC genes. Nature 332, 161-164 (1988).
32. Wong, W., Morris, P. J. & Wood, K. J. Syngeneic bone marrow expressing a single donor class I MHC molecule permits acceptance of a fully allogeneic cardiac allograft. Transplantation 62, 1462-1468 (1996).
33. Davies, J. D., Leong, L. Y., Mellor, A., Cobbold, S. P. & Waldrnann, H. T cell suppression in transplantation tolerance through linked recognition. J. Immunol. 156, 3602-3607 (1996).
34. Wofsy, D., Mayes, D. C., Woodcock, J. & Seaman, W. E. Inhibition of humoral immunity in vivo by monoclonal antibody to L3T4: studies with soluble antigens-in intact mice. J. Immunol. 135, 1698-1701 (1985).
35. Benjamin, R. J. & Waldmann, H. Induction of tolerance by monoclonal antibody therapy. Nature 320, 449451 (1986).
36. Qin, S. et al. Induction of tolerance in peripheral T cells with monoclonal antibodies. Eur. J. Immunol. 20, 2737-2745 (1990).
37. Dossetor, J. B., MacKinnon, K. J., Gault, M. H. & MacLean, L. D. Cadaver kidney transplants. Transplantation 5, Suppl: 844-853 (1967).
38. Morris, P. J., Ting, A. & Stocker, J. Leukocyte antigens in renal transplantation. 1. The paradox of blood transfusions in renal transplantation. Med. J. Aust. 2, 1088-1090 (1968).
39. Opelz, G. & Terasaki, P. I. Improvement of kidney-graft survival with increased numbers of blood transfusions. N. Engl. J Med. 299, 799-803 (1978).
40. Scully, R., Qin, S., Cobbold, S. & Waldmann, H. Mechanisms in CD4 antibody-mediated transplantation tolerance: kinetics of induction, antigen dependency and role of regulatory T cells. Eur. J. Immunol. 24, 2383-2392 (1994).
41. Karim, M., Steger, U., Bushell, A. R. & Wood, K. J. The role of the graft in establishing tolerance. Front. Biosci. 7, e129-154 (2002).
42. O'Farrell, A.-M., Liu, Y., Moore, K. W. & Mui, A. L.-F. IL-10 inhibits macrophage activation and proliferation by distinct signaling mechanisms: evidence for Stat3-dependent and -independent pathways. EMBO J. 17, 1006-1018 (1998).
43. Gulbenkian, A. R. et al. Interleukin-5 modulates eosinophil accumulation in allergic guinea pig lung. Am. Rev. Respir. Dis. 146, 263-266 (1992).
44. Peto, R. et al. Design and analysis of randomized clinical trials requiring prolonged observation of each patient. II. analysis and examples. Br. J. Cancer 35,1-39 (1977).
45. Abrams, J. S. et al. Strategies of anti-cytokine monoclonal antibody development: immunoassay of IL-10 and IL-5 in clinical samples. Immunol. Reiv. 127, 5-24 (1992).
46. Waldmann, H. & Cobbold, S. Approaching Tolerance in Transplantation. Int. Arch. Allergy Immunol. 136, 11-22 (2001)

1-59. (canceled)

60. A method of treating a condition in an animal mediated by an immune response which comprises administering to said animal an antibody directed at a cell surface antigen selected from the group consisting of CD4, CD8, CD 154, LFA-1, CD80, CD86 and ICAM-1, and a non-cellular protein antigen to generate a population of regulatory T-lymphocytes.

61. The method of claim 60, further comprising reactivating said population of regulatory T-lymphocytes by further administration to the animal of the non-cellular protein antigen and transplanting said organ or tissue whilst said population of regulatory T-lymphocytes is activated.

62. The method of claim 60, wherein the condition is an autoimmune condition or rejection of a transplanted organ or tissue.

63. The method of claim 62, wherein the autoimmune condition is selected from the group consisting of rheumatoid arthritis, multiple sclerosis, insulin dependent diabetes mellitus and inflammatory bowel disease.

64. The method of claim 60, wherein the animal is also treated with an immunosuppressive agent or other adjunctive therapy.

65. The method of claim 64, wherein the animal is treated with a sub-therapeutic dose of an immunosuppressive agent.

66. The method of claim 65, wherein the other adjunctive therapy comprises treatment with an anti-CD8 antibody.

67. The method of claim 60, wherein the animal is a human.

68. The method of claim 60, wherein the antibody is an anti-CD4 antibody.

69. The method of claim 68, wherein the antibody is a non-depleting anti-CD4 antibody.

70. The method of claim 60, wherein the non-cellular protein antigen is administered parenterally.

71. The method of claim 60, wherein the antibody is administered to the animal in a dose in the range 0.25 to 25 mg/kg.

72. The method of claim 60, wherein the antibody is administered to the animal in a dose in the range 5 to 10 mg/kg.

73. The method of claim 71, wherein 1, 2, 3, 4, or 5 such doses are administered to the animal.

74. The method of claim 72, wherein 1, 2, 3, 4, or 5 such doses are administered to the animal.

75. The method of claim 60, wherein the non-cellular protein antigen is selected from the group consisting of human gamma globulin, equine gamma globulin and ovalbumin.

76. An ex vivo method for generating a population of regulatory T lymphocytes comprising culturing T cells with an antibody directed at a cell surface antigen selected from the group consisting of CD4, CD8, CD154, LFA-1, CD80, CD86 and ICAM-1 in the presence of cells that present either alloantigen or a non-cellular protein antigen.

77. The method of claim 76, wherein the non-cellular protein antigen is selected from the group consisting of human gamma globulin, equine gamma globulin and ovalbumin.

78. The method of claim 76, wherein the T cells are taken from a recipient animal and the cells that present alloantigen are either cells taken from a donor animal or cells pulsed with antigen taken from a donor animal.

79. A method of suppressing rejection of an organ or tissue transplant in a recipient animal comprising the following steps:
(a) taking a sample of T cells from the recipient animal;
(b) taking a sample of alloantigen from a donor animal, said donor animal being the source of the organ or tissue being transplanted;
(c) exposing said sample of T cells to said sample of alloantigen in the presence of an antibody directed at a cell surface antigen selected from the group consisting of CD4, CD8, CD 154, LFA-1, CD80, CD86 and ICAM-1 to generate a population of regulatory T lymphocytes;
(d) administering to the recipient animal said population of regulatory T-lymphocytes ex vivo.

80. The method of claim 79, wherein said population of regulatory T-lymphocytes is administered to the recipient animal after transplant of an organ or tissue.

81. The method of claim 79, wherein the animal is a human.

82. The method of claim 79, wherein the antibody is an anti-CD4 antibody.

83. The method of claim 82, wherein the antibody is a non-depleting anti-CD4 antibody.

84. Regulatory T lymphocytes produced by the method of claim 76.
